(19) United States
(12) Patent Application Publication
(10) Pub. No.: US 2008/0146951 A1
(43) Pub. Date: Jun. 19, 2008

(54) REGIONAL CEREBRAL VOLUME FLOW USING QUANTITATIVE MAGNETIC RESONANCE ANGIOGRAPHY

(76) Inventors: Meide Zhao, Oswego (IL); Sepideh Amin-Hanjani, Chicago, IL (US); Sean Ruland, Chicago, IL (US); Anthony Curcio, River Forest, IL (US); Lauren Ostergren, Ithaca, NY (US); Fady T. Charbel, River Forest, IL (US)

Correspondence Address: SCHWEGMAN, LUNDBERG & WOESSNER, P.A., P.O. BOX 2938, MINNEAPOLIS, MN, 55402, US

(21) Appl. No.: 11/858,424

(22) Filed: Sep. 20, 2007

Related U.S. Application Data
(60) Provisional application No. 60/846,000, filed on Sep. 20, 2006.

(51) Int. Cl. A61B 5/026

(52) U.S. Cl. 600/504

(57) ABSTRACT

A method and system for evaluating the cerebral circulation of a patient is described. The arterial network of the brain is partitioned into a plurality of different regions where each region is supplied with blood by particular cerebral arteries. Volume flows into the regions are calculated from the measured volume flows of the cerebral arteries using quantitative magnetic resonance angiography. The cerebrovascular circulation of an individual patient may therefore be evaluated for the presence of disease by comparing the regional blood flows of the patient to normal values as obtained from population data.

### RELATED APPLICATIONS

This application is based upon, and claims priority to, previously filed provisional application Ser. No. 60/846,000, filed on Sep. 20, 2006. The provisional application is hereby incorporated by reference. This application is also related to U.S. patent application Ser. Nos. 09/400,365, 11/032,306, 11/049,618 and 11/324,126, the disclosures of which are incorporated by reference in their entirety. The methods and systems described herein may be combined or incorporated into any of the systems described in the aforementioned applications.

### BACKGROUND

Quantification of blood flow to the brain may be useful for distinguishing patients at risk for cerebral ischemia caused by hemodynamic compromise. Hemodynamic assessment by quantitative magnetic resonance angiography (QMRA) has been utilized to identify patients at high risk for stroke and guide treatment decisions. QMRA has been used to determine the total cerebral blood flow (CBF), the effect of age and gender on the total CBF, distribution of CBF in the Circle of Willis, cerebral auto-regulation, and to evaluate various cerebrovascular disorders. The range of blood flow for a healthy individual vessel in the brain, however, can be quite diverse due to inherent vascular anatomy and/or anatomic variations in the Circle of Willis. A decreased volume flow in an individual vessel may not necessarily be caused by vascular disease. An effective decision-making paradigm based upon hemodynamic assessment requires an algorithm that accounts for both anatomic variations and assessment of volume flows in distal vessels (regional CBF). However, current methods for assessing regional CBF can be technically difficult, time-consuming, and may subject the patient to ionizing radiation.

### BRIEF DESCRIPTION OF THE DRAWINGS

FIG. 1 depicts an exemplary system for calculating regional cerebral blood flows.

FIG. 2A shows six slice planes showing where the flow measurements were made for the six neck vessels

FIG. 2B shows seven slice planes showing where the flow measurements were made for the seven head vessels

FIG. 3 is a NOVA 3D surface rendering showing where the measurement was made for BA and its flow direction determined by NOVA software

FIG. 4 illustrates a partition tree of the cerebral circulation

FIG. 5 shows the relative contribution of volume flow in each of eleven regions to its parent region

### DESCRIPTION

Described herein is a method and system for evaluating the cerebral circulation of a patient. Regional cerebral volume flow is derived using flows of selected vessels as measured from quantitative magnetic resonance angiography (QMRA). The arterial network of the brain is partitioned into a plurality of different regions where each region is supplied with blood by particular cerebral arteries. In an exemplary embodiment, the cerebral circulation is partitioned into twelve different regions, with volume flows into the regions being calculated from the measured volume flows of fifteen cerebral arteries. Cerebral regional blood flows calculated in this manner have been shown to exhibit relatively little variation from individual to individual in the absence of cerebrovascular disease. The cerebrovascular circulation of an individual patient may therefore be evaluated for the presence of disease by comparing the regional blood flows of the patient to normal values as obtained from population data.

### Blood Flow Quantification by QRMA

In magnetic resonance imaging, the spins of specific nuclei (usually hydrogen nuclei) in a tissue are excited by radiofrequency (RF) pulses in the presence of an applied static magnetic field in a selected direction, the magnitude of which is made to spatially vary in a defined time sequence. The precessional frequencies of the excited spins vary in relation to the magnitude of the applied magnetic field and thereby produce a precessional signal from which the spatial locations of the spins can be derived. By applying one or more excitation RF pulses and a specific sequence of linear spatial variations in the applied magnetic field, referred to as gradient pulses, the resulting precessional signal can be interpreted as a carrier waveform amplitude modulated by the Pourier transform of the spatial distribution of spin density in a selected portion of the tissue. The carrier waveform in this case is a complex sinusoid at the spin resonance frequency with no gradient applied (i.e., the Larmor frequency of the spin species). Transformation from the spatial frequency domain, referred to as k-space, to the image position domain can be accomplished by inverse Fourier transforming the k-space signal which is generated after demodulation of the precessional signal. The k-space signal is thereby transformed to a spin density function in position space which can be used to generate an image where the intensity of an image pixel varies in accordance with the magnitude of the spin density function at the pixel location. In order to image a selected volume of interest (VOI) in the body, an MRI data set is acquired which is made up of a plurality of slices derived from a two-dimensional (2D) spin density function or a plurality of slabs derived from a three-dimensional (3D) spin density function. As the term is used herein, “image” should be taken to mean either an actual visual representation or the data from which such a representation could be rendered. Similarly, a “pixel” or “voxel” should be taken to mean either a discrete element of an actual 2D or 3D visual representation, respectively, or the corresponding element of a 2D or 3D object from which such a representation could be rendered.

The time sequence of RF excitation and gradient pulses may be manipulated so that the spin density function derived from the k-space signal is dependent upon other parameters in addition to spin density, such as the spin-lattice relaxation time constant T₁ or the spin-spin relaxation time constant T₂. The time constant T₁ relates to the time required for spins to recover longitudinal magnetization after an excitation pulse, the longitudinal magnetization being necessary for the generation of an FID signal following an excitation pulse. A pulse sequence may be designed so that spins with a shorter T₁ are weighted more heavily in the spin density function, and a so-called T₁ weighted image may be derived from such a spin density function. The time-of-flight (TOF) method of imaging blood flow in tissue involves the use of repeated excitation pulses timed so that blood flowing from an unexcited region into the region excited by the pulses has a greater longitudinal magnetization than the stationary tissue in the excited region. The moving blood thus mimics a tissue with a short T₁ and produces an enhanced spin signal. TOF imaging may be used to selectively image blood vessels owing to the moving blood contained within the vessels.

Blood flow may be imaged and quantified by another technique, quantitative magnetic resonance angiography (QMRA). The k-space signal from the excited spins is a complex signal in which the real and imaginary components modulate the carrier waveform in phase quadrature. Ideally, inverse Fourier transformation of the k-space signal results in a purely real spin density function. Certain artifacts may cause the spin density function to have both real and imaginary parts, but this problem can be circumvented in normal imaging by varying the image pixel or voxel intensity in accordance with the magnitude of the spin density function to create a so-called magnitude image. In QMRA, on the other hand, a bipolar gradient pulse is used to cause flowing spins to acquire a phase which is proportional to the velocity of the spins in the direction of the gradient. After such phase-encoding of velocity, the phase can be extracted from the spin density function to measure the magnitude of blood flow. The extracted phase can also be used to construct an image where the pixel or voxel intensity varies with the phase of the spin density function at the location of the pixel or voxel, called a phase image. A phase image derived from a k-space signal derived after application of an appropriate through-plane bipolar gradient pulse can thus provide a visual representation of the magnitude of blood flow through the plane of the image. Measurement of the phase at a particular point in the image provides a measurement of the velocity of the blood flow. By selecting an anatomical plane perpendicular to the direction of a blood vessel and applying a through-plane gradient, a phase signal proportional to the velocity of blood flow can be combined with a measurement of the vessel diameter to quantify the amount of blood flowing through the vessel.

### Exemplary System Description

FIG. 1 shows the components of an exemplary system appropriate for implementing the methods for assessing cerebral circulation as described herein. The system generates phase contrast magnetic resonance (PCMR) images (as well as possibly other data) which may be in a format such as DICOM (Digital Imaging and Communication in Medicine) which is a standard protocol for sending, receiving and storing medical images. The system includes an MRI system 101 as a source of the DICOM images generated for a particular patient. Additional imaging systems which use different imaging modalities such as CT (Computed Tomography), ultrasound, or conventional X-ray angiography may also be used to generate DICOM images or data. The imaging system or systems are connected via a local network to a DICOM storage device 120 such as a PACS (Picture Archiving and Communication System) that is widely used in the clinical and radiological environment. The PACS system provides the functionality of retrieving DICOM images in response to queries received over the network. The system also includes one or more user computers 130 where all or part of the data presentation and/or decision-making software resides. The user computer includes an input device (e.g., keyboard) and a device for displaying the data and/or images to a user (e.g., a monitor). The user computers 130 are connected with the PACS 120 via either a LAN (Local Area Network) or via the internet. (Alternatively, the PACS system may be incorporated into the user computer 130.) The decision making software can take several forms such as a stand-alone application, an added-on feature of existing medical imaging or management software, or an embedded applet within a web browser. The communication and interaction between user computers 130 and the PACS 120 may be based on internet protocols such as HTTP or HTTPS, depending on the requirement of security.

The system may be used to make quantitative flow measurements of the extracranial and intracranial arteries using quantitative phase contrast magnetic resonance angiography. In this technique, an axial 3-D time-of-flight MR angiography is first performed to obtain a three dimensional surface rendering of the vasculature, including the circle of Willis, and a perpendicular cut to the axis of the desired vessel is generated when a vessel is picked in the 3D image. A phase contrast MR scan is then performed using a bipolar gradient pulse directed transverse to the perpendicular cut to generate a phase image which represents blood velocity through the perpendicular cut. Blood flow may then be calculated based upon the cross-sectional area of the selected vessel. The system may be programmed to perform these steps automatically. In order to evaluate the cerebrovascular circulation of a patient, the system is employed to measure blood flow through selected arteries that supply defined cerebral regions and to calculate the blood flow to the regions. By comparing the calculated regional blood flows to normal ranges (e.g., as derived from population data), a patient's hemodynamic status may be determined.

Described below are techniques for deriving regional cerebral volume flow using flows of selected vessels as measured from quantitative magnetic resonance angiography (QMRA). In one particular study discussed below, volume flow rates in fifteen major cerebral arteries were measured on a retrospectively-gated fast 2D phase-contrast magnetic resonance angiography (MRA) obtained in 83 healthy adult volunteers (age range, 24-74 years; mean 42). The arterial network of the brain was partitioned into twelve different regions, in which volume flows were calculated from the measured volume flows of the fifteen cerebral arteries. The twelve regions in the cerebral circulation were identified and formed into a partition tree and the mean volume flow for each region was determined by using vessel flows from QMRA. The mean volume flows of the fifteen arteries and the twelve regions were then calculated. The mean total cranial flow and the mean total CBF were 949±158 ml/min and 695±113 ml/min respectively. The mean volume flows for the anterior and posterior circulation were 483±87 ml/min and 212±34 ml/min respectively. The relative contributions of the volume flows in the eleven regions to their parent regions were obtained. The mean volume flows in the individual arteries and the regions with age were also calculated. The mean volume flows for the female group were significantly lower than the male group (p<0.001) for the two common carotids and the cranial circulation and left/right extracranial circulation. However, the intracranial circulation was not different between genders.

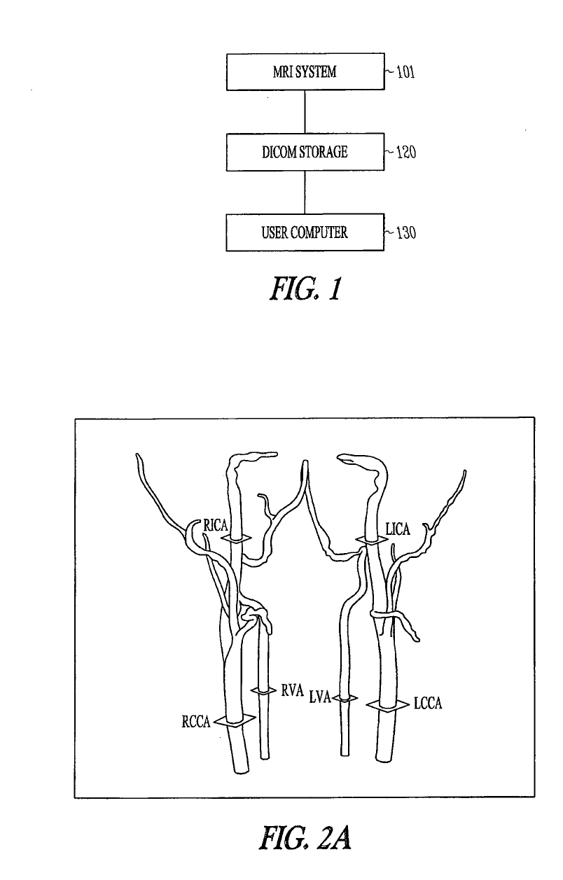

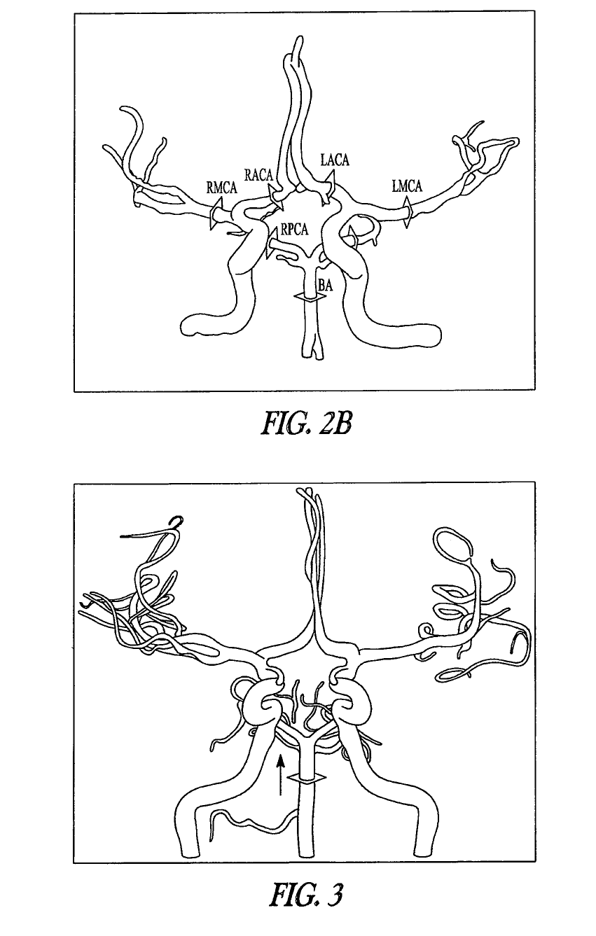

In the example study, the volume flows to specific brain areas, i.e., regional cerebral volume flow was determined using volume flows of individual vessels obtained from QMRA. QMRA was performed on either a 1.5 T or 3 T GE MRI imager (General Electric, Milwaukee, Wis.). The MR volume flow measurements were calculated from NOVA software (VasSol, Inc., Chicago, Ill.) on a separate PC workstation. The protocol utilized a retrospectively gated fast 2D phase contrast sequence. The measured vessels include six vessels in the neck, seven vessels in the head, and the left and the right posterior communicating artery (LPCOM and RPCOM) if present. The six vessels in the neck include the left and the right common carotid arteries (LCCA and RCCA), the left and the right internal carotid arteries (LICA and RICA), and the left and the right vertebral arteries (LVA and RVA). FIG. 2A depicts the locations of the neck vessel measurements. The seven vessels in the head include the basilar artery (BA), the left and the right middle cerebral arteries (LMCA and RMCA) M1 segment, the left and the right anterior cerebral arteries (LACA and RACA) A1 segment, and the left and the right posterior cerebral arteries (LPCA and RPCA) P2 segment. FIG. 2B depicts the locations of the intracranial vessel measurements. The measurements for both the volume flow and the flow direction for each vessel as well as the vascular anatomy of both head and neck were assessed on a PC workstation using the partition algorithm described below. The volume flow represented the average blood flow expressed in milliliters per minute (ml/min). The flow direction and the slice plane perpendicular to the longitudinal axis of a vessel segment showing where the measurement was made were shown in a rotating 3D surface rendering (FIG. 3).

### Partition Algorithm

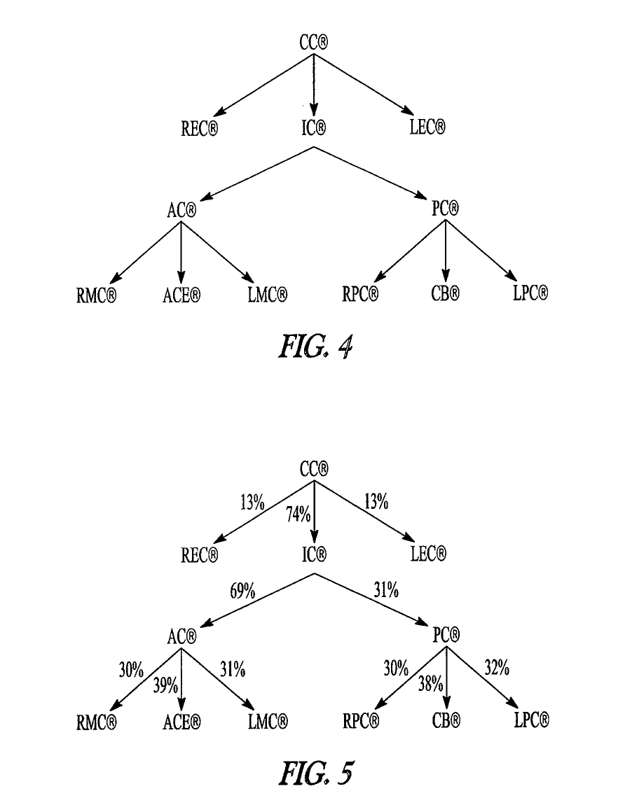

Cranial circulation (CC^{R}) is the circulation in the cranial region (where “R” in CC^{R} and other circulation designations stands for region). CC^{R} consists of the right extracranial circulation (REC^{R}) the left extracranial circulation (LEC^{R}), and the intracranial circulation (IC^{R}). IC^{R} consists of the anterior circulation (AC^{R}) and the posterior circulation (PC^{R}). AC^{R} consists of the right middle cerebral region (RMC^{R}), the left middle cerebral region (LMC^{R}), and the anterior cerebral region (ACE^{R}). PC^{R} consists of the right posterior cerebral region (RPC^{R}), the left posterior cerebral region (LPC^{R}), and the cerebellar-basilar region (CB^{R}). The partitions of the cranial circulation were formed into a partition tree (FIG. 4). A parent region can have one or more than one sub-regions. CC^{R} is the parent region for LEC^{R}, REC^{R}, and IC^{R}. IC^{R} is the parent region for AC^{R} and PC^{R}. AC^{R} is the parent region for LMC^{R}, RMC^{R}, and ACE^{R}. PC^{R} is the parent region for LPC^{R}, RPC^{R}, and CB^{R}.

The volume flow to each of the twelve regions was calculated as follows. The volume flow to CC^{R} was determined by summing the volume flows of the four inlet vessels to the brain, i.e., LCCA, RCCA, LVA, and RVA. The volume flow to IC^{R} was determined by summing the volume flows of the four inlet vessels to IC^{R}, i.e., LICA, RICA, LVA, and RVA, which is the mean total CBF. The volume flow to LEC^{R} (or REC^{R}) was determined by subtracting the volume flow of LICA (or RICA) from the volume flow of LCCA (or RCCA). The volume flow to AC^{R} was obtained as follows: summing the volume flows of LICA and RICA; subtract the volume flow of any fetal PCA or posterior directed PCOM flow. Alternatively, anterior directed PCOM flow would be added. The volume flow to PC^{R} was obtained by subtracting the volume flow to AC^{R} from the volume flow to IC^{R}. The volume flow to LMC^{R}/RMC^{R}/LPC^{R}/RPC^{R} was simply set to be the volume flow of LMCA/RMCA/LPCA/RPCA respectively. The volume flow to ACE^{R} was calculated by summing the volume flows of LACA and RACA (valid only for healthy volunteers, otherwise flow direction should be considered). The volume flow to CB^{R} was determined by subtracting the sum of the volume flows to LPC^{R} and RPC^{R} from the volume flow to PC^{R}.

### Demonstration Study

In order to verify that regional blood flows calculated using the partition algorithm described above are relatively consistent for different individuals in the absence of cerebrovascular disease, a demonstration study was performed. Ninety-two healthy adult volunteers (age range, 21-74 years; mean age, 42 years; 43 men, 49 women) without history of cerebrovascular disease underwent QMRA of head and neck vessels. Quantitative volume flows were expressed as means ±standard deviations. Differences in age between men and women were tested by using a Student independent t-test. Paired Student t-test was used to determine the differences between volume flows for the left and right arteries (or regions). The effect of age on volume flows for individual vessels and regions was evaluated by using linear regression analysis. In all tests, p-value<0.05 was considered statistically significant. All analyses were performed with Analyse-it (ver. 1.71, Analyse-it Software, LTD, England, UK).

Of ninety-two subjects, nine were excluded due to subject motion (n=1), missed RACA measurement (n=1), and missed PCOM measurements (either LPCOM or RPCOM or both PCOMs, n=7). Eighty-three subjects (age rage, 24-74 years; mean age, 42 years; 40 men, 43 women) had adequate QMRA studies and were included in the data analysis. Among the 83 subjects, seven subjects had unilateral fetal PCAs (8%) and three bilateral fetal PCAs (4%); one persistent trigeminal artery (1%); one absent RACA (1%); 28 subjects had no PCOMs (34%), 22 had both PCOMs (26%), 33 had only one PCOM (40%). The flow directions in the PCOMS were posterior in all but one subject. Nine subjects had very short M1 segment (11%) where the volume flows of the two M2 branches were measured and summed to derive the volume flow of the M1 segment.

The mean volume flows of the fifteen arteries and the twelve regions are shown in Table 1 and Table 2 respectively. The mean volume flows were: CC^{R} 949±158 ml/min; IC^{R} 695±113 ml/min; AC^{R} 483±87 ml/min; and PC^{R} 212±34 ml/min. The relative volume flow contribution in each of eleven regions to its parent region is shown in FIG. 5. The relative contribution from the three sub-regions to CC^{R} was 74%±9 for IC^{R}, 13%±5 for LEC^{R}, and 13%±5 for REC^{R}. The relative contribution to IC^{R} from its two sub-regions was 69%±3 for AC^{R} and 31%±3 for PC^{R}. The relative contribution to AC^{R} from its three sub-regions was 31%±4 for LMC^{R}, 30%±3 for RMC^{R}, and 39%±6 for ACE^{R}. The relative contribution to PC^{R} from its three sub-regions was 32%±5 for LPC^{R}, 30%±5 for RPC^{R}, and 38%±9 for CB^{R}.

Table 3 shows the age difference of mean volume flows for individual arteries and regions. Overall, both total cranial flow (CC^{R}) and total cerebral flow (IC^{R}) declined with age (p<0.02), from 1047±178 ml/min (CC^{R}) and 788±102 ml/min (IC^{R}) for the youngest group (aged 24-30 years) to 905±129 ml/min (CC^{R}) and 621±71 ml/min (IC^{R}) for the oldest group (aged 61-74 years).

Table 4 shows the gender difference of the mean volume flows for individual arteries and regions. The mean volume flows for the female group were lower than the male group (p<0.001) for the total cranial circulation (CC^{R}), the two common carotids (LCCA and RCCA), and the left/right extracranial circulation (LEC^{R} and REC^{R}). However, the intracranial circulation (IC^{R}) was not different between genders.

### Tables

TABLE 1
Volume flows (mean ± SD, ml/min) in the fifteen arteries

| LCCA | LVA | LICA | LMCA | LACA | LPCA | LPCOM | BA |
|---|---|---|---|---|---|---|---|
| 389 ± | 96 ± | 264 ± | 150 ± | 85 ± | 66 ± | 17 ± | 131 ± |
| 73 | 38 | 52 | 31 | 26 | 14 | 25 | 40 |

| RCCA | RVA | RICA | RMCA | RACA | RPCA | RPCOM |
|---|---|---|---|---|---|---|
| 381 ± | 83 ± | 252 ± | 145 ± | 80 ± | 63 ± | 15 ± |
| 79 | 32 | 52 | 27 | 28 | 14 | 22 |

TABLE 2
Volume flows (mean ± SD, ml/min) in the
twelve regions of cerebral circulation

| CC^{R} | LEC^{R} | AC^{R} | LMC^{R} | ACE^{R} | LPC^{R} |
|---|---|---|---|---|---|
| 949 ± | 126 ± | 483 ± | 150 ± | 187 ± | 66 ± |
| 158 | 61 | 87 | 31 | 50 | 14 |

| IC^{R} | REC^{R} | PC^{R} | RMC^{R} | CB^{R} | RPC^{R} |
|---|---|---|---|---|---|
| 695 ± | 129 ± | 212 ± | 145 ± | 82 ± | 63 ± |
| 113 | 63 | 34 | 27 | 26 | 14 |

TABLE 3
Volume flows of either individual arteries
or regions decreased with age

| Artery or | | The Youngest | The Oldest |
|---|---|---|---|
| Region | P-value | Group (ml/min) | Group (ml/min) |
| LCCA | p < .01 | 423 ± 86 | 349 ± 51 |
| LVA | p < .01 | 125 ± 35 | 69 ± 39 |
| LICA | p < .03 | 296 ± 45 | 221 ± 49 |
| BA | p < .03 | 164 ± 25 | 118 ± 15 |
| LACA | p < .01 | 101 ± 28 | 67 ± 20 |
| CC^{R} | p < .02 | 1047 ± 178 | 905 ± 129 |
| IC^{R} | p < .02 | 788 ± 102 | 621 ± 71 |
| AC^{R} | p < .02 | 559 ± 74 | 433 ± 52 |
| PC^{R} | p < .03 | 229 ± 32 | 188 ± 23 |

TABLE 4
Gender differences in volume flows of either individual arteries
or regions. F ≦ M (or F ≧ M) indicates that the volume flow for
female group is lower (or higher) than the volume flow for male group

| | Volume Flow | Volume Flow | | |
|---|---|---|---|---|
| Artery or | (ml/min) | (ml/min) | | |
| Region | (Female) | (Male) | Hypotheses | p-value |
| LCCA | 366 ± 64 | 414 ± 73 | F ≦ M | p < .001 |
| RCCA | 345 ± 56 | 419 ± 82 | F ≦ M | p < .0001 |
| CC^{R} | 897 ± 139 | 1006 ± 161 | F ≦ M | p < .001 |
| LEC^{R} | 99 ± 53 | 154 ± 56 | F ≦ M | p < .0001 |
| REC^{R} | 96 ± 41 | 164 ± 65 | F ≦ M | p < .0001 |

In this study, the arterial network of the head was partitioned into twelve different regions. Regional volume flows were calculated using the measured volume flows of the fifteen cerebral arteries as obtained by QMRA. Based upon these measurements in subjects without history of cerebrovascular disease, the standard deviations of the relative contribution of the anterior and posterior circulation (AC^{R} and PC^{R}) to the intracranial circulation (IC^{R}) are small (3%). The standard deviations of the relative contribution of the left and right middle cerebral territory (LMC^{R} and RMC^{R}) to the anterior circulation (AC^{R}) are also small (4% and 3% respectively). Unlike the volume flows of individual vessels that are subject to wide variability based upon anatomic variations in the circle of Willis (15), these regional volume flows may provide a more reliable measure of hemodynamic status.

A limitation of the present study was that the volume flows of the ophthalmic and middle meningeal arteries were assumed to be negligible. While this assumption may be reasonable for healthy volunteers, those volume flows could be significant in some patients with occlusive intracranial cerebrovascular disease and, therefore, must be taken into account in the calculation of the regional volume flows. Additionally, diseases like moya-moya may be poorly evaluated by regional volume flow, as large vessels are typically replaced by multiple intracranial and extracranial collaterals which are not amenable to flow measurement and would not be accounted for by the partitioning model. For the patient with an EC-IC bypass (18), the volume flow at the bypass must be measured and included in the regional volume flow calculation.

### OTHER EMBODIMENTS

As discussed above, the regional volume flows of the brain can be calculated using QMRA. The relative contribution of the various sub-regions to the parent region along with the regional volume flows provides useful information in the hemodynamic evaluation for patients with cerebrovascular disease. Regional blood flow quantification may be implemented in any of the systems described in U.S. patent application Ser. Nos. 09/400,365, 11/032,306, 11/049,618 and 11/324,126 by measuring blood flows in selected blood vessels and calculating the regional blood flows in accordance with the algorithm described above. Regions in addition to, or instead of, the regions defined in the example study discussed above may be defined. For example, regions for the right and left hemispheres of the brain may be defined and total blood flow to each of those regions may be calculated from measurements of blood flow in the individual vessels that supply the regions. In one specific embodiment, the algorithm is implemented in a computer program to perform the regional blood flow calculations automatically. In this embodiment, an operator selects the appropriate vessels for QMRA and provides an identification of those vessels to the program. After measurement of the blood flows in the individual vessels, the program performs the regional blood flow calculations and displays the results to the operator. The results may be displayed in graphic form such as in FIG. 5 to show the relative contribution of volume flow in each of the regions to its parent region.

The invention has been described in conjunction with the foregoing specific embodiments. It should be appreciated that those embodiments may also be combined in any manner considered to be advantageous. Also, many alternatives, variations, and modifications will be apparent to those of ordinary skill in the art. Other such alternatives, variations, and modifications are intended to fall within the scope of the following appended claims.

What is claimed is:

1. A method for evaluating the cerebrovascular circulation of a patient, comprising:
partitioning the cerebral circulation into a plurality of cerebral regions that are supplied with blood by particular arteries;
measuring the blood flow through each of the particular arteries;
calculating the blood flow into each of the plurality of cerebral regions from the blood flow measurements; and,
comparing the calculated cerebral regional blood flows to normal ranges.

2. The method of claim 1 wherein the blood flow through each of the particular arteries is measured by QMRA.

3. The method of claim 1 wherein partition of the cerebral circulation into a plurality of cerebral regions is a partition tree that includes the cranial circulation region (CC^{R}) that consists of the right extracranial circulation (REC^{R}), the left extracranial circulation (LEC^{R}), and the intracranial circulation (IC^{R}).

4. The method of claim 3 wherein the IC^{R} consists of the anterior circulation (AC^{R}) and the posterior circulation (PC^{R}).

5. The method of claim 4 wherein the AC^{R} consists of the right middle cerebral region (RMC^{R}), the left middle cerebral region (LMC^{R}), and the anterior cerebral region (ACE^{R}).

6. The method of claim 5 wherein the PC^{R} consists of the right posterior cerebral region (RPC^{R}), the left posterior cerebral region (LPC^{R}), and the cerebellar-basilar region (CB^{R}).

7. The method of claim 6 wherein the volume flow to CC^{R} is determined by summing the volume flows of the LCCA, RCCA, LVA, and RVA.

8. The method of claim 7 wherein the volume flow to ICR is determined by summing the volume flows of the LICA, RICA, LVA, and RVA.

9. The method of claim 8 wherein the volume flow to LEC^{R} is determined by subtracting the volume flow of LICA from the volume flow of LCCA and the volume flow to REC^{R} is determined by subtracting the volume flow of RICA from the volume flow of RCCA.

10. The method of claim 9 wherein the volume flow to AC^{R} is obtained by summing the volume flows of LICA and RICA and subtracting the volume flow of any fetal PCA or posterior directed PCOM flow.

11. A system for evaluating the cerebrovascular circulation of a patient, comprising:
means for identifying particular arteries supplying blood to a plurality of selected cerebral regions;
means for measuring blood flow in the identified arteries; and,
means for calculating the blood flow to each of the plurality of selected cerebral regions.

12. The system of claim 11 wherein the blood flow through each of the particular arteries is measured by QMRA.

13. The system of claim 11 wherein partition of the cerebral circulation into a plurality of cerebral regions is a partition tree that includes the cranial circulation region (CC^{R}) that consists of the right extracranial circulation (REC^{R}), the left extracranial circulation (LEC^{R}), and the intracranial circulation (IC^{R}).

14. The system of claim 13 wherein the IC^{R} consists of the anterior circulation (AC^{R}) and the posterior circulation (PC^{R}).

15. The system of claim 14 wherein the AC^{R} consists of the right middle cerebral region (RMC^{R}), the left middle cerebral region (LMC^{R}), and the anterior cerebral region (ACE^{R}).

16. The system of claim 15 wherein the PC^{R} consists of the right posterior cerebral region (RPC^{R}), the left posterior cerebral region (LPC^{R}), and the cerebellar-basilar region (CB^{R}).

17. The system of claim 16 wherein the volume flow to CC^{R} is determined by summing the volume flows of the LCCA, RCCA, LVA, and RVA.

18. The system of claim 17 wherein the volume flow to IC^{R} is determined by summing the volume flows of the LICA, RICA, LVA, and RVA.

19. The system of claim 18 wherein the volume flow to LEC^{R} is determined by subtracting the volume flow of LICA from the volume flow of LCCA and the volume flow to REC^{R} is determined by subtracting the volume flow of RICA from the volume flow of RCCA.

20. The system of claim 19 wherein the volume flow to AC^{R} is obtained by summing the volume flows of LICA and RICA and subtracting the volume flow of any fetal PCA or posterior directed PCOM flow.
